(19) United States
(12) Patent Application Publication
(10) Pub. No.: US 2008/0167622 A1
(43) Pub. Date: Jul. 10, 2008

(54) Needle Stick Injury Safety Device

(76) Inventor: Paul Adolf Noppen, New South Wales (AU)

Correspondence Address: ABELMAN, FRAYNE & SCHWAB, 666 THIRD AVENUE, 10TH FLOOR, NEW YORK, NY, 10017, US

(21) Appl. No.: 11/886,411

(22) PCT Filed: Mar. 16, 2006
(86) PCT No.: PCT/AU2006/000386

§ 371 Date: Sep. 13, 2007

(30) Foreign Application Priority Data
Mar. 16, 2005 (AU) 2005901284

(51) Int. Cl. A61M 5/32

(52) U.S. Cl. 604/192

(57) ABSTRACT

A reusable, hand held, safety device (10) for preventing needle stick injury has an elongated body (12) that includes a first opening (14) at a first end thereof. The first opening (14) is adapted to receive therein a capped needle (100) and to retain the cap (101) upon withdrawal of the needle (104) therefrom. There are also first wall means (18) extending outwardly from the first opening (14) sufficiently to provide a barrier to prevent needle stick injury when a user grips the elongated body (12) in one hand and uses the other hand to insert the needle (104) into the cap (101) retained in the first opening (14) to thereby recap the needle. There are also means (22) for releasing the recapped needle from the first opening (14). The elongated body (12) also includes a second opening (16) at a second end thereof. The second opening (16) is adapted to snugly receive and retain therein a vacuum charged, biological fluid collection container (102). There are also second wall means (20) extending outwardly from the second opening (16) sufficiently to provide a barrier to prevent needle stick injury when the user grips the elongated body (12) in one hand and uses the other hand to insert a needle through a vacuum sealing membrane of the container (102) retained in the second opening (16). The releasing means (22) can also release the container (102) from the second opening (16).

### FIELD OF INVENTION

The present invention relates to a reusable, hand held, safety device for preventing needle stick injury to persons using needles for collecting, transferring and/or injecting biological fluids, such as blood.

### BACKGROUND OF THE INVENTION

Needle stick of the invention are a common and significant problem experienced by many healthcare workers. The problem is particularly concerning where the injury is caused by hollow-bore, “dirty” needles, such as hypodermic syringe needles, winged butterfly needles, and intravenous cannulas, which, by their exposure to body fluids, have the potential to transmit infectious disease causing (pathogenic) agents, such as blood borne viruses. Among all healthcare workers, nursing personnel appear to sustain the highest rate of “dirty” needle stick injuries arising, mainly, from poor technique in using hypodermic syringe needles, and this is especially prevalent in tertiary teaching hospitals.

Whilst the introduction of self-retracting safety syringes and a reluctance to use winged butterfly needles has led to a reduction in the incidence of needle stick injury, the higher cost of purchasing such safety syringes, due to their more complex structure and consumable nature, imposes a burden on the budget of many medical institutions and healthcare providers, especially tertiary teaching hospitals. Less expensive, conventional needle and syringe assemblies are still commonly used in most hospitals and by a large number of doctors and other medical professionals in private practice as part of their clinical activities. The threat of needle stick injury posed by the requirement to recap the exposed, hollow bore needle after use has prompted the present inventor to develop a reusable, hand held device which can be used to protect a user's hand against injury when the needle is being recapped and which has other functions that facilitate safer needle manipulation and disposal practices.

### SUMMARY OF THE INVENTION

According to the present invention, there is provided a reusable, hand held, safety device for preventing needle stick injury, comprising an elongated body including a first opening at a first end thereof, the first opening being adapted to receive therein a capped needle and to retain the cap upon withdrawal of the needle therefrom, first wall means extending outwardly from the first opening sufficiently to provide a barrier to prevent needle stick injury when a user grips the elongated body in one hand and uses the other hand to insert the needle into the cap retained in the first opening to thereby recap the needle, and means for releasing the recapped needle from the first opening.

Preferably, the elongated body includes a second opening at a second end thereof, the second opening being adapted to snugly receive and retain therein a vacuum charged, biological fluid collection container, second wall means extending outwardly from the second opening sufficiently to provide a barrier to prevent needle stick injury when the user grips the elongated body in one hand and uses the other hand to insert a needle through a vacuum sealing membrane of the container retained in the second opening, and means for releasing the container from the second opening.

It is preferred that the elongated body includes a passageway interconnecting the first and second openings, and that the releasing means includes a release member that is slidable through the passageway, and when slid in a first direction therethrough collides with the recapped needle to cause its release from the first opening, and when slid in a second direction therethrough collides with the container to cause its release from the second opening.

In a preferred form, the releasing means further includes a control member that is connected to the release member, the control member being engaged with the elongated body and movable to cause the release member to slide through the passageway, the control member including a grip knob for enabling manual movement of the control member in the first and second directions.

Preferably, the elongated body is a tubular body, and the control member includes a collar that is slidably movable upon an outer surface portion of the tubular body, the tubular body including a longitudinal slot through which the control member is connected to the release member.

It is preferred that the tubular body includes a longitudinal track along which a carriage of the control member slides upon manual movement of the control member.

The first and second wall means may be discs having a general shape of a dish. Preferably, each disc is formed integrally with the elongated body.

### BRIEF DESCRIPTION OF THE DRAWINGS

FIG. 1 is a perspective view of a preferred safety device of the present invention,

FIG. 2 is a top view of the safety device of FIG. 1,

FIG. 3 is a sectional end view through III-III of the safety device of FIG. 2,

FIG. 4 is a sectional side view through IV-IV of the safety device of FIG. 2,

FIG. 5 is an enlarged sectional side view of a first end of the safety device shown in FIG. 4,

FIG. 6 is an enlarged sectional side view of a second end of the safety device shown in FIG. 4,

FIG. 7 is an exploded perspective view of the safety device of FIG. 1,

FIG. 8 is a partly sectional side view of the safety device of FIG. 1 being used to retain a capped hypodermic syringe needle,

FIG. 9 is a view similar to that of FIG. 8 but showing the safety device being used to release the capped hypodermic syringe needle therefrom,

FIG. 10 is a partly sectional side view of the safety device of FIG. 1 being used to retain a capped multisampling blood collection needle and a holder,

FIG. 11 is a view similar to that of FIG. 10 but showing the safety device being used to release the capped multisampling blood collection needle therefrom,

FIG. 12 is a partly sectional side view of the safety device of FIG. 1 being used to retain a biological fluid collection container,

FIG. 13 is a view similar to that of FIG. 12 but showing the safety device being used to release the biological fluid collection container therefrom,

FIG. 14 is a perspective view of a “dirty” hypodermic syringe needle being inserted into a cap therefor retained in the safety device of FIG. 1, with the hand gripping the device being behind a first protective disc, and

FIG. 15 is a perspective view of a hypodermic syringe needle being used to fill with blood a biological fluid collection container retained in the safety device of FIG. 1, with the hand gripping the device being behind a second protective disc.

### MODES FOR CARRYING OUT THE INVENTION

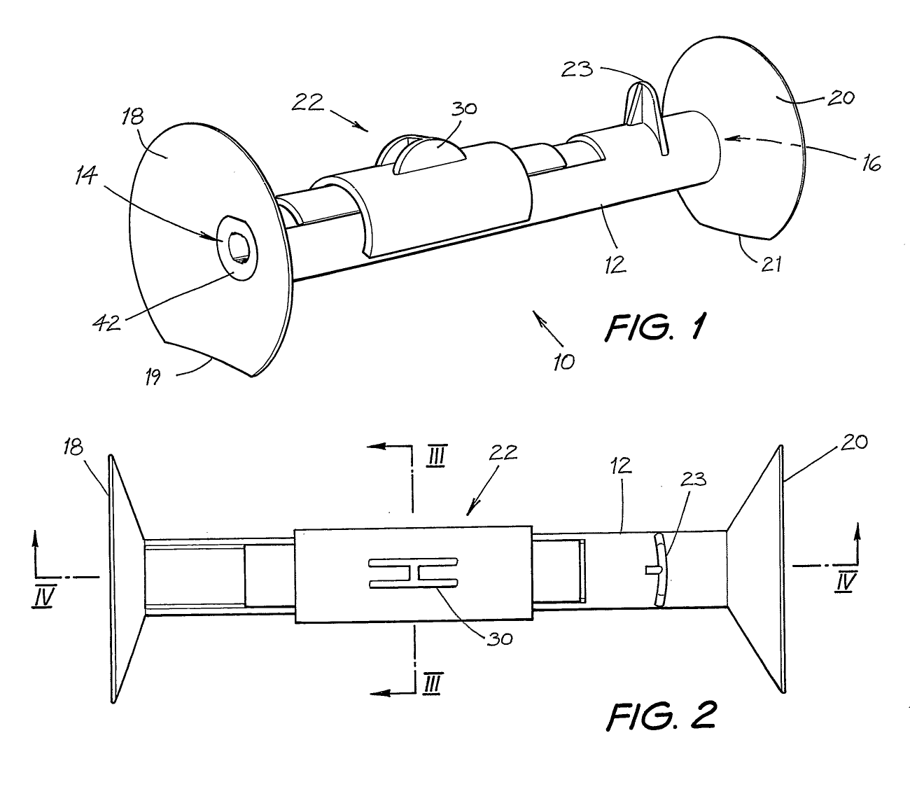

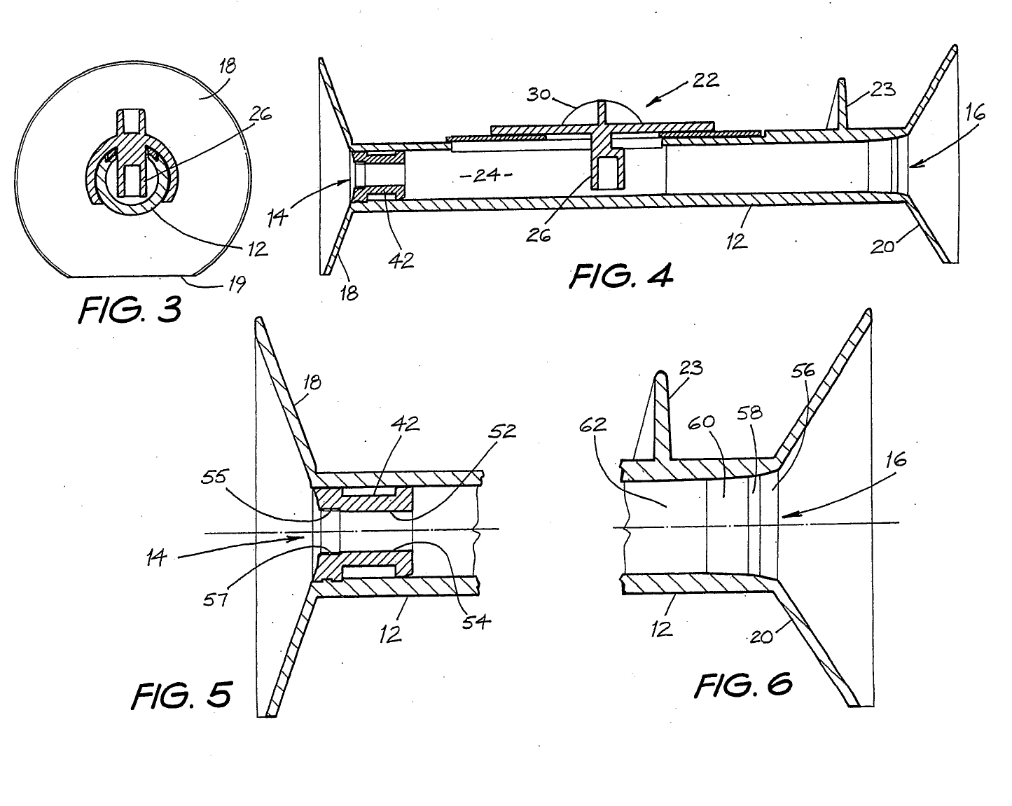

The reusable, hand held, safety device 10 shown in full and in part in FIGS. 1 to 15 has a tubular body 12 (although the body may be of any suitably elongated shape), a first opening 14 at a first end thereof, a second opening 16 at a second end thereof, a first wall means 18 extending outwardly from the first opening 14, a second wall means 20 extending outwardly from the second opening 16, releasing means 22 slidably engaged with the tubular body 12, and a thumb grip 23 extending outwardly from the tubular body 12.

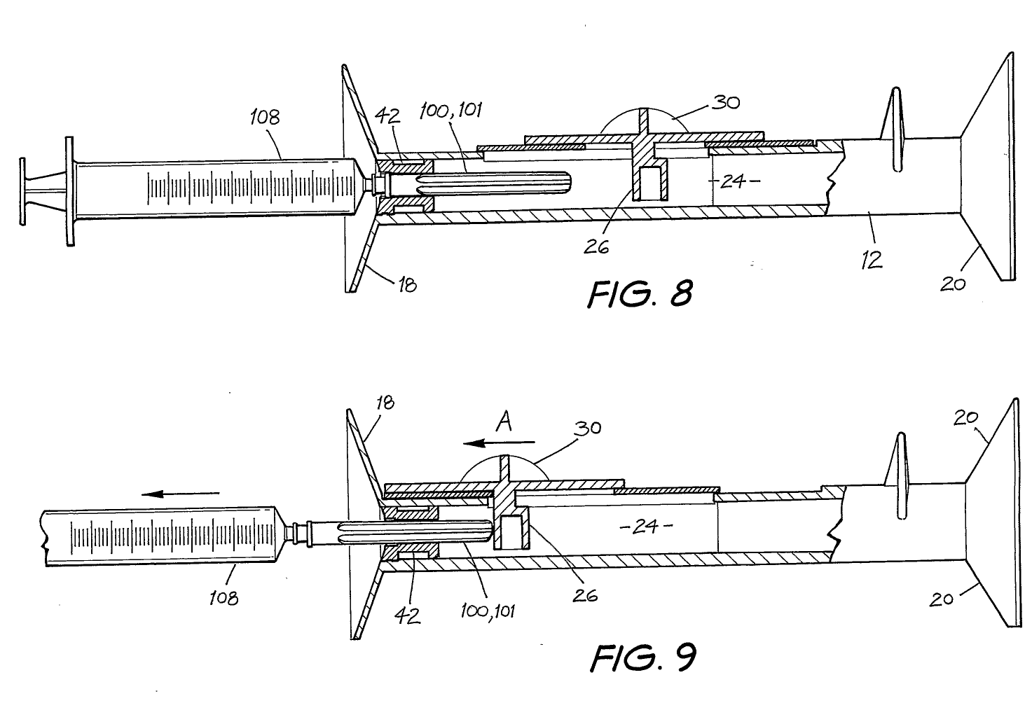

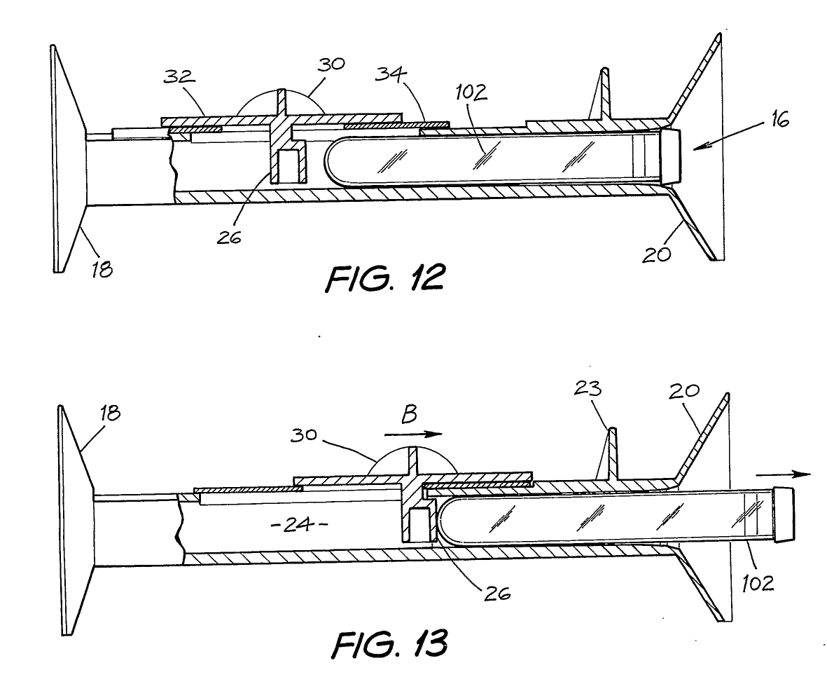

The first opening 14 is adapted to receive therein a capped needle 100 (as shown in FIGS. 8 to 11) and to retain the cap 101 upon withdrawal of the needle 104 therefrom. The second opening 16 is adapted to snugly receive and retain therein a vacuum charged, biological fluid collection container 102 (as shown in FIGS. 12 and 13).

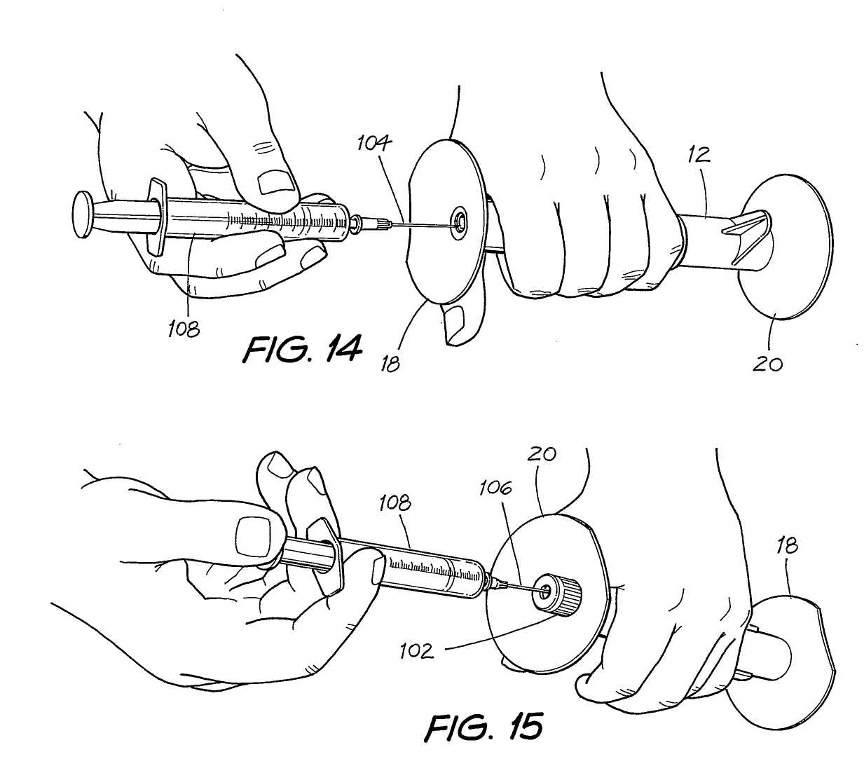

The first wall means 18 is a disc having the shape of a dish with a cut away minor segment that creates a linear edge portion 19 that prevents the device 10 rolling from its position when supported by its linear edge portion 19. The outer rim of the disc 18 is sufficiently distant from the first opening 14 that it provides a barrier to prevent needle stick injury when (as shown in FIG. 14) a user grips the tubular body 12 in one hand and uses the other hand to insert a needle 104 into a cap 101 retained in the first opening 14 for recapping the needle 104.

The second wall means 20 is also a disc having the shape of a dish with a cut away minor segment that creates a linear edge portion 21. The outer rim of the disc 20 is sufficiently distant from the second opening 16 that it provides a barrier to prevent needle stick injury when (as shown in FIG. 15) a user grips the tubular body 12 in one hand and uses the other hand to insert a needle 106 through a vacuum sealing membrane of a vacuum charged, biological fluid collection container 102 retained in the second opening 16.

The releasing means 22 is, by virtue of its slidable engagement with the tubular body 12, able to release the recapped needle 100 from the first opening 14 (as shown in FIG. 9) and release the container 102 from the second opening 16 (as shown in FIG. 13).

It will be apparent to skilled persons in the art, however, that, where a safety device is required only for the purposes of uncapping a needle, retaining the cap, and recapping the needle, the second opening and second wall means (as described above) may be omitted from the device, and the releasing means need only be able to release the recapped needle from the first opening.

The tubular body 12 of the safety device 10 has a passageway 24 interconnecting the first and second openings 14, 16. Slidable through the passageway 24 is a release member 26 of the releasing means 22. When the release member 26 is slid in a first direction (as shown by arrow A in FIG. 9) through the passageway 24, the release member 26 collides with the recapped needle 100 to cause its release from the first opening 14. When the release member 26 is slid in a second direction (as shown by arrow B in FIG. 13) through the passageway 24, the release member 26 collides with the container 102 to cause its release from the second opening 16.

Connected to the release member 26 is a control member 28 of the releasing means 22. The control member 28, which engages the tubular body 12, is movable to cause the release member 26 to slide through the passageway 24. The control member 28 includes a grip knob 30 and a collar 32. The control member 28 slidably engages a carriage 34 of the releasing means 22, which carriage 34 slidably engages the tubular body 12. The carriage 34 has a longitudinal slot 36 formed therethrough.

The tubular body 12 includes a longitudinal track 38 formed as a depression in the outer surface of the tubular body 12, and there is a longitudinal slot 40 formed therethrough.

The grip knob 30 can be gripped or otherwise pressed by a user to cause movement of the control member 28 in the first and second directions.

The collar 32 of the control member 28 is thereby slidably movable upon both an outer surface portion of the tubular body 12 and the carriage 34.

The carriage 34 is slidable along the track 38, and the slot 36 of the carriage 34 is superimposed on the slot 40 of the track 38. The slot 36 is shorter than the slot 40, but throughout the range that the carriage 34 may be slid along the track 38 (from the end of the track 38 nearest the first end of the body 12 to the end of the track 38 nearest the second end of the body 12) there is a continuous opening through both slots 36, 40 through which a stem portion 41 passes that connects the control member 28 to the release member 26. In the preferred embodiment shown in the drawings, the control member 28 is integrally connected, say, by injection moulding, to the release member 26.

The relative arrangement of the slots 36, 40 and track 38 are such that, when a user moves the grip knob 30 in a first direction (see arrow A in FIG. 9) to its furthermost allowable extent, the release member 26 collides with the recapped needle 100 and pushes it out from the first opening 14 sufficiently that it can be dropped into a “sharps” disposal container. Similarly, when a user moves the grip knob 30 in a second direction (see arrow B in FIG. 13) to its furthermost extent allowable by the relative arrangement of the slots 36, 40 and track 38, the release member 26 collides with the biological fluid collection container 102 and pushes it out from the second opening 16 sufficiently that it can be dropped into a disposal container dedicated to that purpose.

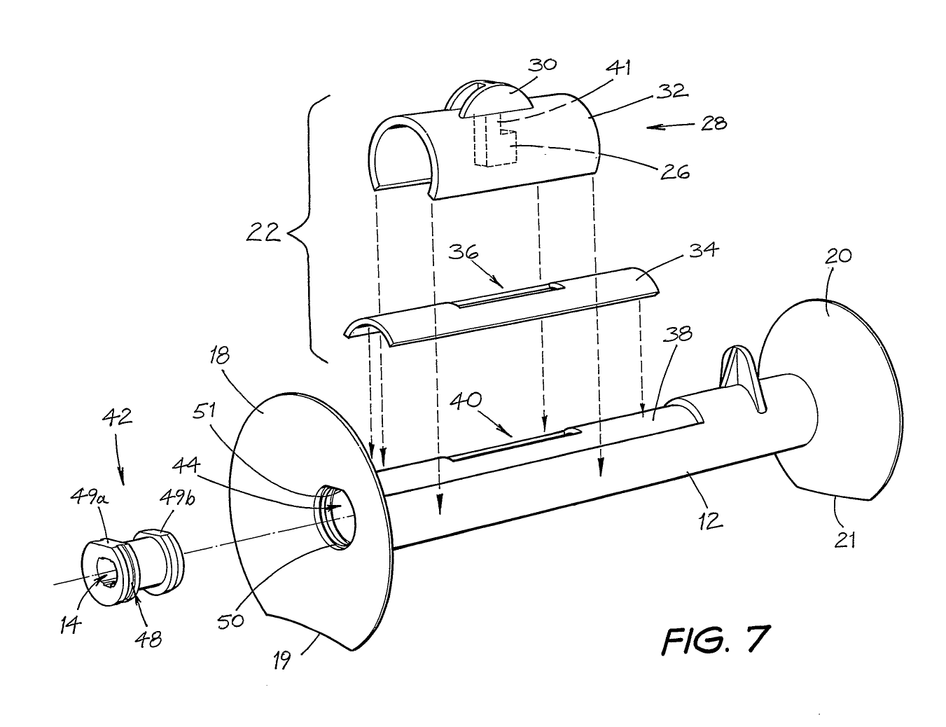

The first opening 14 into which a capped needle 100 can be received and the cap 101 thereof retained is defined by a cap receiving insert 42 shown isolated as part of FIG. 7. The insert 42 fits tightly (as shown in FIG. 5) into a correspondingly shaped cavity 44 at the first end of the tubular body 12. The insert 42 has an outer part circumferential groove 48 that is securely engaged by an inner part circumferential nib 50 of the cavity when the insert 42 is correctly located or keyed within the cavity. The insert 42 also has flat, outwardly facing segments 49a,49b, segment 49a being continuous with a side wall of the groove 48 such that the groove 48 opens into the segment 4 a at the ends of its part circumferential path. The cavity 44 has a flat, inwardly facing segment 51 of complementary size to the segments 49a,49b so that, when the insert 42 is correctly located within the cavity 44, the flat segment 49a is keyed against the flat segment 51. In this way, the insert 42 is prevented from rotating within the cavity 44 when any rotating force is applied thereto during the drawing of a needle 104 out from a cap 101 received in the opening 14. When so located, the outermost end of the insert 42 is flush with the outermost extent of the cavity 44. The insert 42 defines an opening 14 that is tapered inwardly and would be entirely cylindrical in cross-section were it not for a pair of opposed top and bottom, inner planar walls 52, 54 that, although themselves inwardly tapered, interrupt the cylindrically tapered shape and extend to the innermost end of the insert 42. The planar walls 52, 54 do not extend to the outermost end of the insert 42 but terminate at respective steps 55,57. The steps 55,57 provide a gripping surface for a capped needle 100 and, by gripping the cap 101, provide a resistance to release of the cap 101 from within the insert 42 when the needle 104 is being drawn therefrom.

The first opening 14 is thus able to receive and retain therein a capped needle having a variety of cap shapes.

For instance, the opening 14 can receive and retain the cap 101,110 of both hypodermic syringe needles 104 and multisampling blood collection needles. A hypodermic syringe needle 104 commonly has a cap 101 which includes a cylindrical ring structure at the mouth of the cap, with the remaining portion of the cap 101 being tapered, and so the inwardly tapered shape of the opening 14 is suitably dimensioned to receive therein a capped hypodermic syringe needle 100 by a tight interference fit (as shown in FIG. 8) after a leading part of the tapered portion of the cap 101 has passed through the first opening 14 into the passageway 24, and to retain the cap 101 upon withdrawal of the needle 104 therefrom.

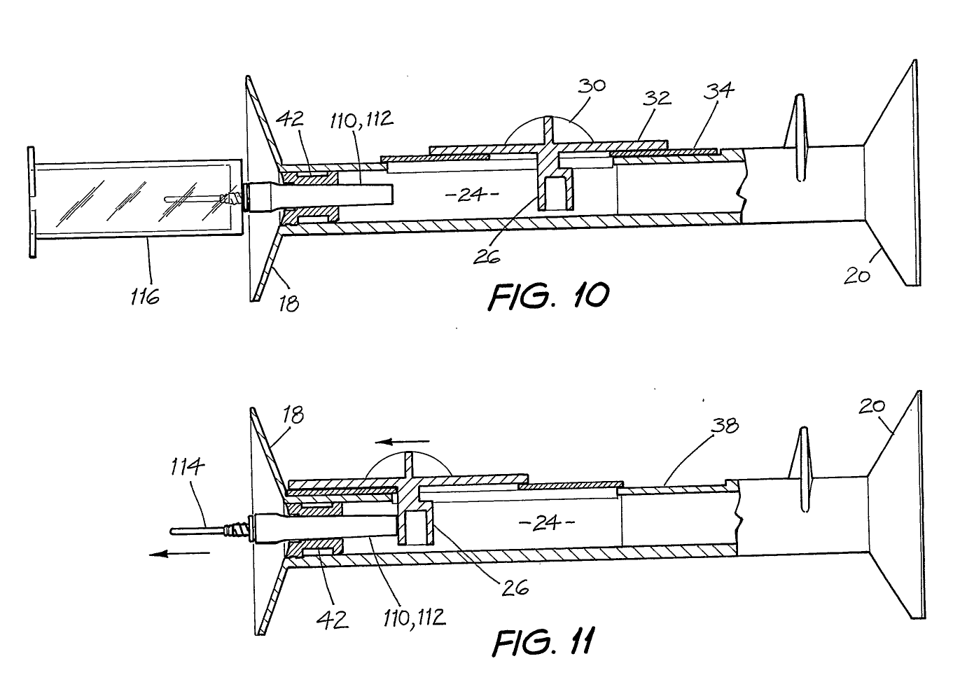

A multisampling blood collection needle commonly has a coloured cap 110 which includes a pair of opposed, outer planar walls that interrupt an otherwise cylindrically tapered portion of the cap, and so the shape of the pair of opposed, inner planar walls 52, 54 of the opening 14 is suitably dimensioned to receive therein a capped multisampling needle 112 by a tight interference fit (as shown in FIG. 10) after a leading part of the tapered portion of the cap 110 has passed through the first opening 14 into the passageway 24, and to retain the cap 110 upon withdrawal of the needle therefrom.

The second opening 16 into which a vacuum charged, biological fluid collection container 102 can be snugly received and retained is defined by four adjacent, separately tapered, cylindrical walls 56, 58, 60, 62 (as shown in FIG. 6) at the second end of the tubular body 12 and which are continuous with the adjacent walls of passageway 24. The separately tapered, outermost walls 56, 58 of the second opening 16 are suitably dimensioned to snugly receive and retain therein a membrane supporting cap of the container 102 by a tight interference fit after the tube of the container 102 has passed through the separately tapered innermost walls 60,62 towards the passageway 24 (as shown in FIG. 12).

The following is a description of the use of the safety device 10 for preventing needle stick injury to a user from a variety of needles.

In the case of a hypodermic syringe needle, the user connects a hypodermic syringe 108 to a capped needle 100 in the usual manner. The user then grips the tubular body 12 of the device 10 and inserts the capped needle 100, to which the syringe 108 is connected, into the first opening 14 until it is firmly received therein. The user then pulls the syringe 108 away from the opening 14 to draw the needle 104, to which the syringe 108 remains connected, out from the cap 101, which is retained in the opening 14. Some rotation of the syringe 108 may be required to assist the pulling of the syringe away from the opening 14. The user carries out the required procedure, following which the user, with one hand, inserts the “dirty” needle 104 firmly back into the cap 101 retained in the opening 14 of the tubular body 12 gripped with the other hand behind the protective disc 18. The releasing means 22 is then operated to drop the capped “dirty” needle 100 into a “sharps” disposal bin.

In the case of a multisampling blood collection needle, the user grips the tubular body 12 of the device 10 and inserts the coloured (usually green or black) capped needle 112 (covering the unsheathed end of the needle) into the first opening 14 until it is firmly received therein. The user then rotates and pulls the partly transparent or white cap away from the retained coloured capped needle 112 to reveal the other sheathed end 114 of the needle. A multiple or single use holder 116 is then screwably connected to the needle so that the sheathed needle end 114 is within the holder 116. The user then pulls the holder 116 away from the opening 14 to draw the unsheathed needle, to which the holder is also connected, out from the coloured cap 110 which is retained in the opening 14. Some rotation of the holder 116 may be required to assist the pulling of the holder away from the opening 14.

The user carries out the required procedure, following which the user, with one hand, inserts the “dirty” unsheathed needle end firmly back into the coloured cap 110 retained in the opening 14 of the tubular body 12 gripped with the other hand behind the protective disc 18. The releasing means 22 is then operated to drop the capped “dirty” needle, to which the holder remains connected, into a “sharps” disposal bin. Alternatively, where it is desired to sterilize the holder, the holder is unscrewed from the “dirty” needle, and the releasing means 22 is then operated to drop the capped “dirty” needle into a “sharps” disposal bin.

In the case of vacuum charged, biological fluid collection containers 102, such as a blood collection bottle which requires a hypodermic syringe needle to fill the bottle with blood of a desired amount, the user inserts a blood collection bottle 102 into the second opening 16 until it is snugly received and retained therein. The user connects a hypodermic syringe 108 to a capped needle 100 in the usual manner. The user then grips the tubular body 12 of the device 10 and inserts the capped needle 100, to which the syringe 108 is connected, into the first opening 14 until it is firmly received therein. The user pulls the syringe 108 away from the opening 14 to draw the needle 104, to which the syringe remains connected, out from the cap 101, which is retained in the opening 14. The user then inverts or rotates the device 10 until it is vertical or standing on the disc 18. The user carries out the required procedure in which the syringe 108 is filled with the desired amount of blood, following which the user, with one hand, inserts the tip of the “dirty” needle 106 into the bottle 102 by (as shown in FIG. 15) piercing the rupturable, vacuum sealing membrane in the cap retained in the opening 16 of the tubular body 12 gripped with the other hand behind the protective disc 20, and injects or fills the bottle 102 with blood of a desired amount. The releasing means 22 is then operated to release the filled bottle 102 from the device 10 for further use or storage. If multiple samples of the blood collected by the syringe needle are required, a desired number of blood collection bottles may be consecutively inserted into the second opening 16 and the procedure repeated. When the bottle filling operation has been completed, the user again inverts or rotates the device 10 and, with one hand, inserts the “dirty” needle firmly back into the cap retained in the opening 14 of the tubular body 12 gripped with the other hand behind the protective disc 18. The releasing means 22 is then operated to drop the capped “dirty” needle into a “sharps” disposal bin.

Various modifications may be made in the design and construction of the safety device without departing from the scope and ambit of the invention.

1. A reusable, hand held, safety device for preventing needle stick injury, comprising an elongated body including a first opening at a first end thereof, the first opening being adapted to receive therein a capped needle and to retain the cap upon withdrawal of the needle therefrom, first wall means extending outwardly from the first opening sufficiently to provide a barrier to prevent needle stick injury when a user grips the elongated body in one hand and uses the other hand to insert the needle into the cap retained in the first opening to thereby recap the needle, and means for releasing the recapped needle from the first opening.

2. The safety device of claim 1 wherein the elongated body includes a second opening at a second end thereof, the second opening being adapted to snugly receive and retain therein a vacuum charged, biological fluid collection container, second wall means extending outwardly from the second opening sufficiently to provide a barrier to prevent needle stick injury when the user grips the elongated body in one hand and uses the other hand to insert a needle through a vacuum sealing membrane of the container retained in the second opening, and means for releasing the container from the second opening.

3. The safety device of claim 2 wherein the elongated body includes a passageway interconnecting the first and second openings, and that the releasing means includes a release member that is slidable through the passageway, and when slid in a first direction therethrough collides with the recapped needle to cause its release from the first opening, and when slid in a second direction therethrough collides with the container to cause its release from the second opening.

4. The safety device of claim 3 wherein the releasing means further includes a control member that is connected to the release member, the control member being engaged with the elongated body and movable to cause the release member to slide through the passageway, the control member including a grip knob for enabling manual movement of the control member in the first and second directions.

5. The safety device of claim 4 wherein the elongated body is a tubular body, and the control member includes a collar that is slidably movable upon an outer surface portion of the tubular body, the tubular body including a longitudinal slot through which the control member is connected to the release member.

6. The safety device of claim 5 wherein the tubular body includes a longitudinal track along which a carriage of the control member slides upon manual movement of the control member.

7. The safety device of claim 1 wherein the first and second wall means are discs having a general shape of a dish.

8. The safety device of claim 7 wherein each disc is formed integrally with the elongated body.
